(19) United States
(12) Patent Application Publication
(10) Pub. No.: US 2008/0195167 A1
(43) Pub. Date: Aug. 14, 2008

(54) CARDIAC PACEMAKERS AND SYSTEMS AND METHODS FOR USING THEM

(76) Inventor: Timothy J. Ryan, Charlottesville, VA (US)

Correspondence Address: Vista IP Law Group LLP, 2040 MAIN STREET, 9TH FLOOR, IRVINE, CA, 92614, US

(21) Appl. No.: 11/966,524

(22) Filed: Dec. 28, 2007

Related U.S. Application Data
(60) Provisional application No. 60/882,976, filed on Dec. 31, 2006.

(51) Int. Cl. A61N 1/368

(52) U.S. Cl. 607/23

(57) ABSTRACT

Systems and methods are provided for determining the pressure-volume relationship for one or more chambers of a heart. An implantable device includes a catheter including a distal end sized for introduction into a chamber of a heart, a pressure sensor for measuring pressure within the chamber, and a resistance sensor for measuring fluid resistance within the chamber. A processor coupled to the catheter obtains pressure data from the pressure sensor and fluid resistance data from the resistance sensor. The processor approximates fluid volume within the chamber as a function of time and determines one or more pressure-volume loops based upon the pressure data and the fluid volume. In one embodiment, the catheter is a lead including a pacing electrode and a controller including the processor delivers pulses to the pacing electrode based upon the pressure-volume loops to deliver electrical therapy to the heart.

This application claims benefit of co-pending provisional application Ser. No. 60/882,976, filed Dec. 29, 2006, the entire disclosure of which is expressly incorporated by reference herein.

### FIELD OF THE INVENTION

The present invention relates generally to implantable devices for measuring pressure and fluid volume within the heart, for example, cardiac pacemakers, e.g., biventricular pacing systems, and, more particularly, to pacemakers and/or pacing systems with resistance and/or pressure sensing capabilities, and to methods for using them.

### BACKGROUND

Implantable cardiac pacemakers are implanted within patients' hearts, e.g., for pacing, sensing and/or defibrillation, e.g., within the right and/or left chambers of the heart. Leads may sense electrical activity of the heart and pacemakers coupled to the leads may provide pacing as needed, depending on the mode of pacing employed. Biventricular pacing has been successfully employed to improve cardiac output in patients with congestive heat failure (“CHF”). This therapy, also known as Cardiac Resynchronization Therapy (“CRT”), is based on the hypothesis that faulty conduction of electrical impulses through the purkinje fibers and myocardium is at least partly to blame for the faulty pumping of the ventricles. Many devices currently available aim to alter the conduction of electrical impulses to the two ventricles to improve pumping efficiency.

Presently, there is no way to continuously directly measure the effects of adjustments of the timing of the electrical impulses without invasive measurements. For example, echocardiography may be used to image the cardiac chambers, yielding a measure of the filling and emptying of the chambers. However, accurate pressure measurements at multiple points in time throughout the cardiac cycle cannot be obtained using echocardiography.

Accordingly, apparatus and methods for pacing the heart would be useful.

### SUMMARY OF THE INVENTION

The present invention is directed to implantable devices for measuring pressure and/or fluid impedance or resistance within the heart, e.g., for recording and/or determining pressure-volume loops. For example, the present invention may be directed to cardiac pacemakers, e.g., biventricular pacing apparatus and systems, and, more particularly, to pacemakers and/or pacing systems with resistance and/or pressure sensing capabilities, and to methods for using them. In exemplary embodiments, pacing leads may be placed in multiple locations within a heart, e.g., within the right ventricle and/or within the left ventricle or into a lateral coronary vein. One or both leads may include pressure sensing and/or fluid resistance sensing, e.g., for fluid volume approximation, which may provide substantially continuous measurement of the Pressure-Volume relationship, e.g., for determining the “PV Loop” for the heart. Such leads and/or pacemakers may provide accurate adjustment of the timing of delivery of electrical pulses to the various chambers of the heart, and/or may enable adjustment of the timing in near real-time, e.g., based on the filling and emptying performance of the cardiac chambers.

In accordance with one embodiment, an implantable device is provided for determining the pressure-volume relationship for a first chamber of a heart. The device may include an elongate member including a proximal end, a distal end sized for introduction into a first chamber of a heart, a pressure sensor on the distal end for measuring pressure within the first chamber, and an impedance or resistance sensor for measuring fluid impedance or resistance within the first chamber. A processor may be coupled to the proximal end of the elongate member for obtaining pressure data from the pressure sensor and fluid impedance or resistance data from the impedance or resistance sensor. The processor may be configured for determining fluid volume data approximating the volume of fluid within the first chamber and/or for determining a pressure-volume relationship for the first chamber based upon the pressure data and the fluid volume data.

In accordance with another embodiment, a system is provided for obtaining data related to the pressure-volume relationship for one or more chambers of the heart. The system may include a first lead including a first proximal end, a first distal end sized for introduction into a body lumen, a pressure sensor on the first distal end for measuring pressure within a first chamber of a heart within which the first distal end is implanted, and a first set of electrodes on the first distal end for measuring impedance or resistance of fluid within the first chamber. A controller may be coupled to the first lead for receiving pressure data and impedance or resistance data between one or more pairs of the first set of electrodes. The controller may include a processor for determining a pressure-volume relationship for the first chamber based upon the pressure and impedance or resistance data. For example, the processor may approximate fluid volume within the first chamber as a function of time using resistance data, and relate the pressure data and approximate fluid volume to determine a pressure-volume loop for the first chamber.

Optionally, the first lead may also include a first pacing electrode for delivering electrical energy to tissue adjacent the first chamber. In this embodiment, the controller may include a pulse generator for delivering electrical energy to the first pacing electrode for pacing the heart based at least in part on the pressure-volume relationship for the first chamber. In addition or alternatively, the system may include a second lead including a second proximal end, a second distal end sized for introduction into a body lumen, and a second pacing electrode on the second distal end for delivering electrical energy to tissue adjacent a second chamber of a heart. In this embodiment, the controller may also be coupled to the second lead such that the pulse generator may deliver electrical energy to the second pacing electrode. In addition or alternatively, in any of these embodiments, the controller may include a transmitter and/or receiver, e.g., for transmitting data, such as the pressure data, impedance or resistance data, approximate fluid volume, and/or pressure-volume relationship, to a remote location, e.g., external to the heart and/or the patient's body, and/or for receiving instructions from a remote location.

In accordance with yet another embodiment, a system is provided for pacing a heart of a patient that includes first and second leads, and a controller. The first lead may include a first proximal end, a first distal end sized for introduction into a body lumen, a pressure sensor on the first distal end for measuring pressure within a first chamber of a heart within which the first distal end is implanted, a first set of electrodes on the first distal end for measuring impedance or resistance of fluid within the first chamber, and a first pacing electrode for delivering electrical energy to tissue adjacent the first chamber. The second lead may include a second proximal end, a second distal end sized for introduction into a body lumen, and a second pacing electrode on the second distal end for delivering electrical energy to tissue adjacent a second chamber of a heart.

The controller may be coupled to the first and second proximal ends, the controller receiving pressure data from the pressure sensor and impedance or resistance data from the plurality of electrodes for determining a pressure-volume relationship for the first chamber. The controller may also include a pulse generator for delivering electrical energy to the first and second pacing electrodes based at least in part upon the determined pressure-volume relationship for the first chamber to deliver electrical therapy to the heart.

In accordance with still another embodiment, a method is provided for biventricular pacing of a heart using first and second leads delivered within the heart. Pressure may be measured within the first chamber and impedance or resistance of fluid within the first chamber may be measured using the first lead. A pressure-volume relationship may be determined for the first chamber based upon the pressure and impedance or resistance measured within the first chamber, and electrical energy may be delivered to electrodes on the first and second leads based at least in part upon the pressure-volume relationship for the first chamber to provide electrical therapy to the heart.

In one embodiment the pressure-volume relationship for the first chamber may be determined by relating the measured resistance to fluid volume within the first chamber as a function of time, and generating a pressure-volume loop based upon the cardiac cycle of the heart based at least in part on the fluid volume of the first chamber as a function of time and the measured pressure. For example, the pressure-volume relationship for the first chamber may be used to determine when the first chamber is optimally filled with blood based upon the pressure-volume loop, and one or more electrodes on the first lead may be activated to cause contraction of the first chamber when the processor determines the first chamber is optimally filled with blood.

In accordance with yet another embodiment, a method is provided for implanting a biventricular pacing system within a heart of a patient. A distal end of a first lead may be delivered through the patient's vasculature into a first chamber of the heart such that a pressure sensor and a first set of electrodes on the distal end are disposed within the first chamber, and a first pacing electrode on the distal end of the first lead may be secured to the myocardium adjacent the first chamber. A distal end of a second lead may be delivered through the patient's vasculature into the heart, and a second pacing electrode on the distal end may be secured to the myocardium adjacent a second chamber of the heart. The first and second leads may be coupled to a controller configured for receiving pressure data from the pressure sensor and impedance or resistance data from the first set of electrodes to determine a pressure-volume relationship for the first chamber. The controller may include a pulse generator for delivering electrical energy to at least one of the first and second pacing electrodes based at least in part upon the determined pressure-volume relationship for the first chamber to deliver electrical therapy to the heart. Optionally, the second lead may include a pressure sensor and a second set of electrodes, and the controller may determine a pressure-volume relationship for the second chamber.

In accordance with still another embodiment, a distribution system and/or method for distributing pacing or PV loop monitoring systems is provided. Generally, a plurality of systems may be provided to health care providers, e.g., doctors, practice groups, hospitals, and the like, without sale. The systems may include one or more leads, PV loop recorders, and/or controllers, such as those described herein. For example, the health care providers may merely rent the system from a source, e.g., a manufacturer, distributor, and the like. The health care providers may provide and/or implant the systems in patients and reimburse the source on a periodic basis for the systems so provided. Alternately, the health care provider or patient may pay a fee to the source of the system for management and collection of data, e.g., by the PV loop recorder. For example, a health care provider may implant a lead and controller in a patient, the controller including a PV loop recorder. The recorder may be coupled to the controller circuitry or may operate independently of the controller circuitry to obtain PV loop data related to the patient. Alternatively, the recorder may be a separate device from the controller implanted within the patient or otherwise coupled to the pressure sensors and resistance electrodes.

Optionally, the source may provide technical support, e.g., using any of the systems and methods described herein, to the health care providers and/or patients. When the systems are removed and/or returned by the health care providers and/or patients to the source, any payments and/or services may be discontinued. Optionally, the source may refurbish or otherwise repair components of the pacing systems, e.g., the controllers, for reuse.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a cross-sectional view of a heart, showing normal conduction pathways within the heart.

FIG. 2 is a cross-sectional view of a heart, showing a first exemplary embodiment of a pacing system implanted within the heart.

FIG. 3 is a side view of a distal end of an exemplary embodiment of a pacing lead that may be included in the pacing system of FIG. 2.

FIG. 4 is a schematic of an exemplary embodiment of a controller that may be provided in a pacing system.

FIG. 5 is a cross-sectional view of a heart, showing a second exemplary embodiment of a pacing system implanted within the heart.

FIG. 6 shows an exemplary idealized pressure-volume loop and an exemplary actual pressure-volume loop for a cycle of a heart.

### DETAILED DESCRIPTION OF THE EXEMPLARY EMBODIMENTS

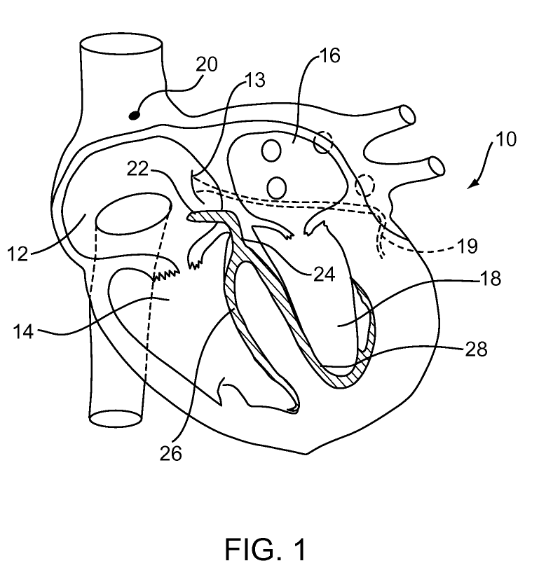

Turning to the drawings, FIG. 1 shows a cross-section of a heart 10, showing the various chambers of the heart, i.e., the right atrium 12, the right ventricle 14, left atrium 16, and left ventricle 18. In addition, FIG. 1 shows conduction pathways of the heart 10, e.g., the sinoatrial (“SA”) node 20, which is the impulse generating tissue in the right atrium 12, and the atrioventricular (“AV”) node 22, which includes the AV bundle or “Bundle of His” 24. The AV bundle 24 splits into two branches, namely the right AV bundle branch 26, which activates the right ventricle 14, and the left AV bundle branch 28, which activates the left ventricle 18. The bundle branches 26, 28 taper out to produce numerous Purkinje fibers, which stimulate individual groups of myocardial cells to contract the chambers of the heart 10.

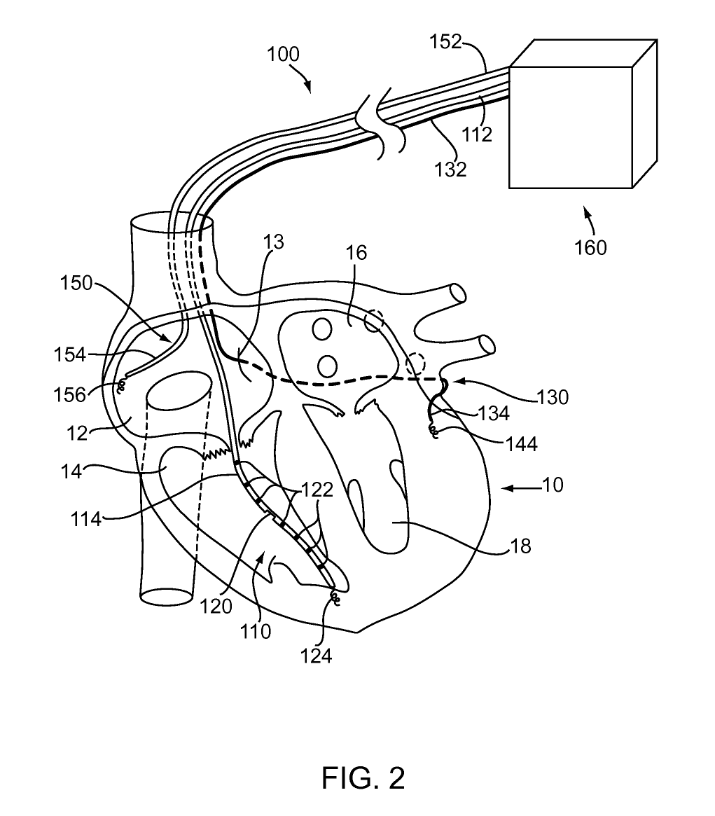

Turning to FIG. 2, an exemplary embodiment of a pacemaker system 100 is shown that may be implanted into a heart, such as the heart 10 of FIG. 1, e.g., for providing biventricular pacing to the heart 10. In addition or alternatively, the system 100 may provide the ability to record and/or determine pressure-volume relationships for one or more chambers of the heart 10. Generally, the system 100 includes one or more catheters or leads, e.g., leads 110, 130, 150, and a controller 160. Optionally, the system 100 may also include one or more additional components, e.g., one or more guidewires, guide catheters, and the like (not shown) for delivering the leads.

The leads 110, 130, 150 may be constructed similar to one another e.g., including one or more electrodes and/or pressure sensors. For example, as shown in FIG. 2, the first lead 110 includes a proximal end 112 coupled to the controller 160, a distal end 114 sized and/or shaped for introduction into a patient's body, and one or more components on the distal end 114. The first lead 110 may have sufficient length to extend from an entry site, e.g., a percutaneous puncture, e.g., in a peripheral vessel of the patient, through the patient's vasculature into the heart 10. The first lead 110 may be formed from plastic, metal, or composite materials, e.g., a plastic material having a wire, braid, or coil core, which may preventing kinking or buckling of the first lead 110 during advancement. For example, the proximal end 112 may be substantially rigid, semi-rigid, or flexible, e.g., having sufficient column strength to facilitate advancing the distal end 114 through a patient's vasculature by pushing on the proximal end 112. The distal end 114 may be substantially flexible or even substantially “floppy,” e.g., to facilitate insertion through tortuous anatomy and/or deep into the patient's vasculature.

Optionally, the first lead 110 may include a lumen (not shown) extending between the proximal and distal ends 112, 114, e.g., to facilitate directing the first lead 110 over a guidewire or other rail (not shown). In addition or alternatively, the first lead 110 may include one or more lumens (also not shown) extending between the proximal and distal ends 112, 114, e.g., for the components on the distal end 114, e.g., one or more wires or other conductors, pressure lumens, and the like, as described further elsewhere herein.

In addition or alternatively, the first lead 110 may include one or more connectors, a handle, and the like (not shown) on the proximal end 112, e.g., for connecting the first lead 110 to the controller 160. For example, the connector may include one or more electrical connectors for coupling electrodes or other electrical components on the distal end 114 to the controller 160 and/or one or more ports communicating with a pressure or other lumen extending between the proximal and distal ends 112, 114.

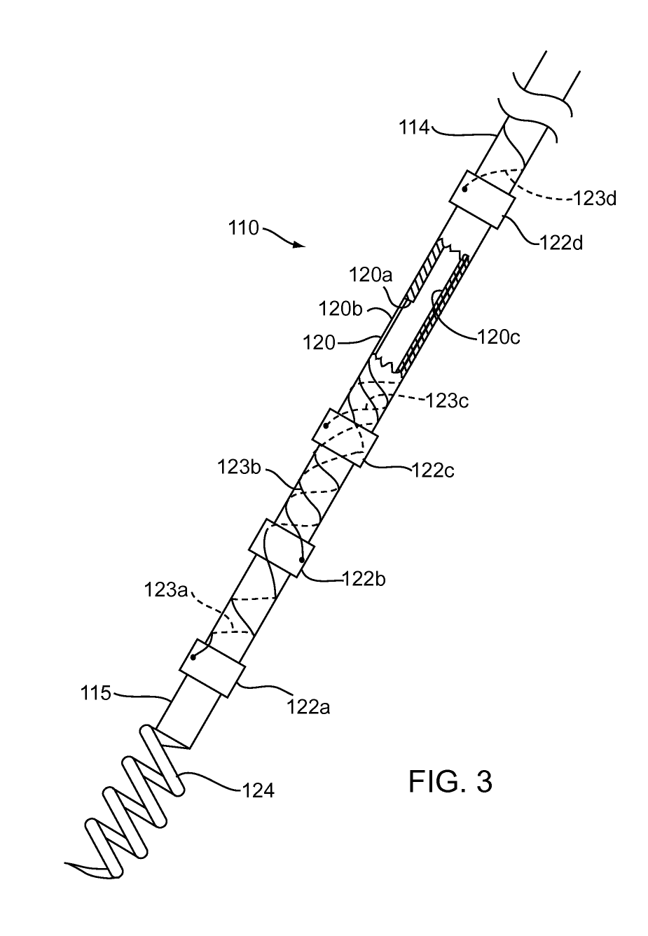

With additional reference to FIG. 3, the distal end 114 may include a pressure sensor 120 for measuring pressure within a first chamber, e.g., the right ventricle 14, a first plurality of electrodes 122 for measuring impedance or resistance of fluid within the right ventricle 14, and a first tip electrode 124 for delivering electrical energy to tissue adjacent the right ventricle 14.

The pressure sensor 120 may include an opening, e.g., a lateral aperture 120a in a wall of the distal end 114, which may be covered with a membrane 120b, e.g., a low-modulus silicone, such as NUSIL 6650, and the like. A pressure lumen 120c may communicate between the aperture 120a and the proximal end 112 of the first lead 110. The pressure lumen 120c may be filled with biocompatible fluid, e.g., an incompressible fluid, such as water, mineral oil, saline, silicone oil, and the like, or a compressible fluid, such as nitrogen, such that variations in pressure on the membrane 120b may be communicated via the pressure lumen 120c to a port or other element (not shown) on the proximal end 112 of the first lead 110.

Alternatively, other pressure sensors may be provided, such as a strain gauge, a piezoresistive transducer, a fiber-optic pressure sensor, and the like may be provided for the pressure sensor 120 instead of the membrane 120b. For example, a piezoresistive microelectronic transducer or absolute strain gauge transducer (not shown) may be attached within or on an inner surface of the wall of the distal end 114 of the lead 14, e.g., as disclosed in U.S. Pat. No. 4,730,619 to Koning et al., the entire disclosure of which is expressly incorporated by reference herein. In such alternatives, one or more wires or other conductors may extend from the pressure transducer 120 to the proximal end 112 of the first lead 110, and the proximal end 112 may include one or more connectors (not shown) for coupling the conductor(s) to the controller 160 (not shown, see FIG. 2).

With continued reference to FIGS. 2 and 3, one or more pacing electrodes 124 may be provided on the distal end 114 of the first lead 110. For example, as best seen in FIG. 3, a tip electrode 124 may be provided on a distal tip 115 of the first lead 110, e.g., having a cork-screw configuration such that the tip electrode 124 may be screwed into the wall of the myocardium. The tip electrode 124 may be electrically coupled to the controller 160 by one or more wires or other conductors (not shown) extending proximally from the distal tip 115, e.g., to one or more connectors (not shown) on the proximal end 112 of the first lead 110.

For example, the tip electrode 124 may be attached to the distal tip 115 of the first lead 110, e.g., by bonding with adhesive, using an interference fit, melting or otherwise fusing the distal tip 115 around or to the tip electrode 124, using mating threads (not shown), and/or using other cooperating connectors. A wire or other conductor (not shown) may be attached to the tip electrode 124, e.g., by welding, soldering, fusing, bonding with adhesive, and the like. The wire may extend through a lumen of the first lead 110 to the proximal end 112 thereof or may be formed along or within the wall of the first lead 110.

Alternatively, the tip electrode 124 may include a rounded, tapered, or other configuration, e.g., if the lead 110 is delivered into a coronary vein or other vessel, rather than a chamber of the heart. Optionally, if the lead 110 is delivered into a coronary vein or other vessel, one or more additional pacing electrodes (not shown) may be provided on the distal end 114 proximal to the tip electrode 124, e.g., for bipolar pacing and the like, if desired. Such electrode(s) may include ring electrodes, wire electrodes, and the like, similar to the impedance or resistance measuring electrodes described elsewhere herein.

In addition, with continued reference to FIGS. 2 and 3, a first set of resistance measuring electrodes 122 may be provided on the distal end 114 of the first lead 110, e.g., a plurality of electrodes 122 spaced apart from one another along the distal end 114 proximal to the tip electrode 124. The electrodes 122 may be spaced apart sufficient distance to facilitate measurement of the resistance of fluid between the electrodes 122, yet sufficiently close such that all of the electrodes 122 are disposed within the first chamber, e.g., the right ventricle 14, when the first lead 110 is delivered into the first chamber. Alternatively, if one or more of the proximal electrodes are disposed outside the first chamber, these proximal electrodes may be ignored by the system 100, e.g., either automatically or based upon instructions from a clinician, as described elsewhere herein.

In the embodiments shown in FIGS. 2 and 3, one or more of the electrodes 122 may be disposed proximal to the pressure sensor 120, while the remainder of the electrodes 122 may be disposed between the pressure sensor 120 and the tip electrode 124. One of the electrodes 122, e.g., proximal electrode 122d in FIG. 3, may be a reference electrode, and another of the electrodes 122, e.g., distal electrode 122a in FIG. 3, may be an active electrode. During use, substantially constant electrical signals may be delivered to the active and reference electrodes, e.g., the proximal and distal electrodes 122d, 122a, and pairs of other electrodes, e.g., electrodes 122b, 122c, may be used to measure resistance between the electrodes 122b, 122c, i.e., due to the resistance of the fluid between the electrodes 122b, 122c. While FIG. 3 only shows a single pair of resistance measuring electrodes 122b, 122c for simplicity, it will be appreciated that multiple pairs of electrodes may be provided along the length of the distal end 114. For example, FIG. 2 includes five electrodes 122 between the proximal and distal electrodes, which may be used to measure resistance between each adjacent pair along the length of the distal end 114, which may be related to fluid volume, as described elsewhere herein.

The electrodes 122 may be formed from metal or other conductive bands disposed around the wall of the distal end 114 and attached thereto, e.g., by an interference fit, bonding with adhesive, crimping around the wall, and the like. Alternatively, the electrodes 122 may be wires or other material wound tightly around the distal end 114, e.g., within a recess, which may also be attached using other methods described herein. In a further alternative, the distal end 114 may include a plurality of tubular segments that be attached between adjacent electrodes 122 to build up the distal end 114 of the first lead 110.

As shown in FIG. 3, one or more wires or other conductors 123 may be coupled to respective electrodes 122 and extend proximally to the proximal end 112 of the first lead 110, e.g., to one or connectors (not shown). As shown, the wires 123 may be wound helically within or along an inner surface of the first lead 110. Alternatively, the wires 123 may extend proximally through one or more lumens (not shown), e.g., through separate wire lumens, or through a single wire lumen, e.g., if the wires 123 are electrically insulated from one another.

Returning to FIG. 2, the second lead 130 includes a second proximal end 132, a second distal end 134 sized for introduction into a body lumen, and a second pacing electrode 144 on the second distal end 134 for delivering electrical energy to tissue adjacent a second chamber of a heart, e.g., the left ventricle 18, as shown. The second lead 130 may be constructed similar to the first lead 110, as described above. In the embodiment shown in FIG. 2, however, the second lead 130 does not include a pressure sensor or resistance measuring electrodes.

The second pacing electrode 144 may be a tip electrode, e.g., having a cork-screw configuration, similar to the tip electrode 124 shown in FIG. 3. Alternatively, for delivery into a coronary vein, such as the lateral coronary vein 19 adjacent the left ventricle 18 (shown in FIG. 1), the second pacing electrode 144 may simply be a rounded tip electrode (not shown). Such an electrode may be maintained within a target vessel, such as the lateral coronary vein 19 simply by friction or interference between the distal end 134 of the second lead 130 and the vessel wall. Optionally, the second pacing electrode 144 or the distal end 134 itself may include one or more ribs or other features on an outer surface thereof (not shown) for enhancing interference or otherwise engaging the distal end 134 within the target vessel, as described elsewhere herein.

With continued reference to FIG. 2, the pacing system 100 may also include a third lead 150, which generally includes a third proximal end 152, a third distal end 154 sized for introduction into a body lumen, and a third pacing electrode 156 on the third distal end 154 for delivering electrical energy to tissue adjacent a third chamber of a heart, e.g., the right atrium 14, as shown. The third lead 150 may be constructed similar to the first lead 110, e.g., as described above, although the third lead 150 generally does not include a pressure sensor or resistance measuring electrodes. The third pacing electrode 156 may be a tip electrode, e.g., having a cork-screw configuration, similar to the tip electrode 124 shown in FIG. 3.

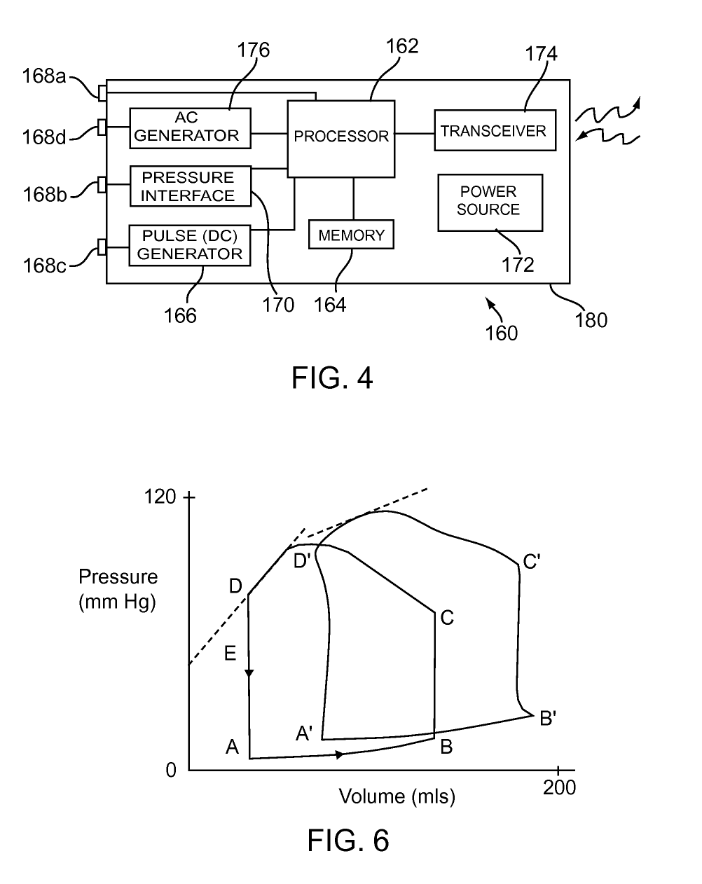

Turning to FIG. 4, with additional reference to FIG. 2, the controller 160 may be coupled to the leads 110, 130, 150 to interface with the various components on the distal ends 114, 134, 154 described above. Generally, the controller 160 may include one or more processors 162, memory 164, and one or more electrical generators, e.g., a direct current (DC) pulse generator 166 and an alternating current (AC) generator 176. For embodiments where the system 100 is intended for recording and/or determining the pressure-volume relationship without pacing, pulse generator 166 may be omitted. Optionally, the controller 160 may also include a pressure interface 170, e.g., for converting hydraulic or pneumatic signals from a pressure sensor (such as pressure sensor 120 of FIG. 2) into electrical signals. For example, the pressure interface 170 may include a plenum or chamber (not shown) within which a strain gauge or other transducer (also not shown) is disposed such that pressure communicated from the pressure sensor 120 may displace or otherwise impose the pressure upon the transducer, which may produce an electrical signal proportional the pressure.

In addition or alternatively, the controller 160 may include a transceiver 174, e.g., one or more transmitters, receivers, and/or other telemetry devices, for communicating with one or more devices or systems external to a patient's body. The controller 160 may also include a power source 172, e.g., one or more batteries, capacitors, and the like, for providing electrical energy to operate the components of the controller 160. Optionally, the controller 160 may include a connector (not shown) for coupling the controller 160 to an external energy source, e.g., an external battery, a charger for recharging the power source 172, and the like, or transformer coils for transcutaneous charging (also not shown).

The components of the controller 160 may be coupled to one another, e.g., using one or more wires, circuit boards, and the like. For example, the components may be mounted to one or more circuit boards, and one or more buses or other conductive pathways may be provided on the circuit board(s) to allow necessary communication and/or data relay between the components.

The components may be provided within a casing 180, which may be substantially fluid tight, e.g., if the controller 160 is to be implanted within a patient's body. The casing 180 may be sufficiently small such that the controller 160 may be implanted within a patient's body, e.g., subcutaneously, or may be carried externally on the patient's body. Alternatively, all or a portion of the processor 162 and/or other components of the controller 160 may be external to the patient, and may communicate with the leads 110, 130, 150 and/or other implanted components of the controller 160, if any, via a catheter, cable, and the like (not shown).

The controller 160 may include one or more connectors 168, which are shown schematically in FIG. 4, for coupling the controller 160 to the leads 110, 130, 150 and/or other external components (not shown). For example, one or more electrical connectors 168a (one shown for simplicity) may be provided for coupling the processor 160 to impedance or resistance measuring electrodes, such as electrodes 122b, 122c shown in FIG. 3. One or more hydraulic or pneumatic connectors 168b may be provided for coupling the pressure interface 170 to one or more pressure sensors, such as pressure sensor 120 shown in FIG. 3. If the pressure sensor 120 provides an electrical output, the pressure interface 170 may be eliminated, and the connector(s) 168b may couple the pressure sensor(s) to the processor 162. One or more electrical connectors 168c may be provided (one shown for simplicity) for coupling the pulse generator 166 to one or more pacing electrodes, such as electrodes 124, 144, 156 shown in FIG. 2. Finally, one or more electrical connectors 168d may be provided (one shown for simplicity) for coupling the AC generator 176 to the reference and active electrodes used for resistance measurement, such as electrodes 122a, 122d shown in FIG. 3.

Although the connectors 168 are shown schematically in FIG. 4, the controller 160 may include separate physical connectors (not shown). Each of the physical connectors may be connected to respective leads 110, 130, 150. Each physical connector may include the appropriate pins, ports, or other electrical, pneumatic, or other connectors to couple the components on the respective lead with the components of the controller 160.

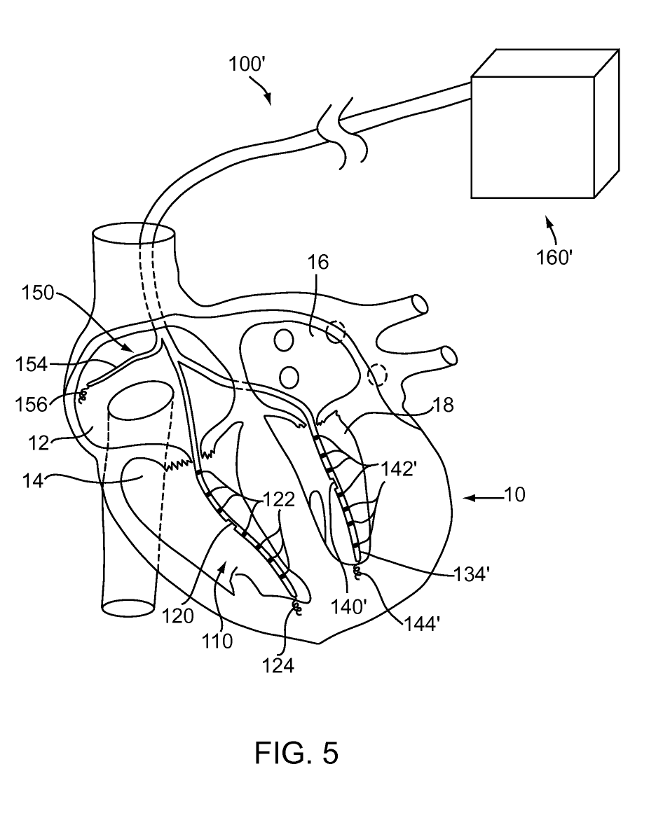

With continued reference to FIG.4, the AC generator 176 may be configured for generating high frequency alternating current, e.g., at one or more frequencies between about one and two kiloHertz (1-2 kHz). For the system 100 shown in FIG. 2, the AC generator 176 may generate signals at a single frequency for delivery to the reference and active electrodes of the first set of electrodes, e.g., electrodes 122d, 122a in FIG. 3. For example, the AC generator 176 may be configured to generate an alternating electrical current of about four microamperes (4 μA) at a frequency of about 1.3 kiloHertz (kHz), the AC generator 176 (and/or processor 162) adjusting the voltage as required to maintain a relatively constant current during impedance or resistance measurement. For the system 100′ shown in FIG. 5, however, the AC generator 176 may generate two separate signals, e.g., one at about 1.3 kHz and another at about 1.6 kHz such that signals may be delivered simultaneously to the first and second sets of electrodes 122, 142,′ as described elsewhere herein. Alternatively, for the system 100′ shown in FIG. 5, the AC generator 176 may generate signals at a single frequency, and the AC generator 176 (or processor 162) may include a switch (not shown) for alternately delivering the signals to the first and second sets of electrodes 122, 142,+ also as described elsewhere herein.

The processor 162 may include one or more processors, subprocessors, and/or other hardware and/or software components (not shown) for controlling operation of other components of the controller 160 and/or for processing data between the other components of the system 100 and/or external components (not shown). For example, the processor 162 may include a general processor for communicating between the components of the controller 160. In addition, the processor 162 may include one or more sensing circuits and/or filters (not shown) for receiving impedance or resistance signals (e.g., via connector 168a), and/or for converting the resistance signals into other data. In addition, the processor 162 may include one or more additional circuits and/or algorithms, e.g., to determine if and when pacing voltage is indicated, i.e., for controlling operation of the pulse generator 172, to monitor, record, and/or transmit system parameters, and the like. The processor 162 may remain fixed once programmed or may be programmable before and/or after implantation of the controller 160, e.g., upon receiving instructions via the transceiver 174, as described elsewhere herein.

Generally, the processor 162 may receiving pressure data from the pressure sensor 120 (via the pressure interface 170), and resistance data from the electrodes 122 to determine a pressure-volume relationship for the first chamber, e.g., the right ventricle 14 shown in FIG. 2. If resistance data is obtained at multiple frequencies (e.g., by delivering different frequency signals to first and second sets of electrodes, the processor 162 may include one or more filters to substantially reduce or eliminate interference between the sets of electrodes. For example, for the embodiment above where a frequency of about 1.3 kHz is used for the electrodes 122, a first band pass filter may be coupled to the electrodes 122 that filters out signals above 1.4 kHz. If a frequency of about 1.6 kHz is used for a second set of electrodes (such as electrodes 142′ in FIG. 5), a second band pass filter may be coupled to the electrodes 142′ that filters out signals below 1.4 kHz. Thus, the filters may reduce the chance of interference between the two frequencies.

When the processor 162 determines that it is appropriate to deliver pacing energy to the patient, the processor 162 may then instruct the pulse generator 166 to deliver electrical signals to one or more of the pacing electrodes 124, 134, 156, e.g., based at least in part upon the pressure-volume relationship for the first chamber to deliver electrical therapy to the heart 10. Generally, the pulse generator 166 may be configured to generate a DC spike or pulse having a desired voltage and duration. The processor 162 may determine the desired voltage and/or duration based upon the resistance of the body pathway, i.e., the electrical passageway through the heart between the active pacing electrodes 124, 134 and the passive electrode 156 through which electrical energy must pass. The processor 162 may determine the desired power to pace the heart, and use Ohm's law to determine the current necessary, adjusting the voltage and duration to achieve the desired power and/or current level. It will be appreciated that other configurations for pacing or otherwise delivering therapeutic electrical energy to the heart may also be used.

In addition, if the controller 160 includes transceiver 174, the controller 160 may cause the transceiver 174 to transmit at least one of the pressure data, resistance data, fluid volume data derived from the resistance data, and/or the pressure-volume relationship to a remote location, i.e., external to the heart 10 and/or the patient's body. In one embodiment, the transceiver 174 may include a wireless transmitter, such as a short range or long range radio frequency (“RF”) transmitter, e.g., using Bluetooth or other protocols. Alternatively, other telemetry may used, such as acoustic or electromagnetic, and the like.

Optionally, the transceiver 174 may also be able to receive communications from a remote source, e.g., a device implanted elsewhere in the patient's body or external to the patient. For example, the transceiver 174 may communicate with an external recorder and/or controller, which may receive data from the controller 160. A clinician or other user may review the data and send instructions back to the controller 174 via the transceiver 174, e.g., modifying pacing or other therapy provided by the system 100 based upon the reviewed data, as described elsewhere herein.

For example, the system 100 may allow data to be recorded, e.g., in real time, and transmit the data at a later time via the transceiver 174. Thus, the controller 160 may be configured to save the data in memory 164 and automatically transmit the data periodically. Alternatively, the controller 160 may periodically poll the transceiver 174 to check for communications from an external source, e.g., such that the controller 160 may only transmit the data when instructed to do so by the external source. In addition or alternatively, the system 100 may allow adjustment of pacing or other electrical therapy based upon characteristics of the pressure-volume loop generated. This adjustment may be automatic, for example, based upon one or more algorithms programmed into the controller 160, or the adjustment may be based upon instructions received via the transceiver 174 from a clinician using an external controller.

In the exemplary embodiment shown in FIG. 2, the system 100 is an implantable biventricular pacemaker with resistance-sensing electrodes and pressure sensing on the right ventricle pacing lead 110. The system 100 may allow generation of PV loops for the right ventricle 14 based upon pressure and resistance data, as desired, and thus may provide a more definite measure of effects of adjustments in pacing or other therapies.

Electrical impedance or resistance of blood or other fluid may be used to approximate volume of fluid within a chamber of the heart, e.g., within the right ventricle 14 for the system 100 shown in FIG. 2. Because the phase shifts involved may be minor, it may not be necessary to measure electrical “impedance” (which includes both a real component and imaginary component, e.g., phase shift), and instead only electrical “resistance” (which includes only the real component). Substantially constant electrical signals may be delivered to two of the electrodes 122, and then respective pairs of resistance measuring electrodes may be activated to determine the electrical resistance of fluid between the pairs, which may be related to fluid volume.

For example, with additional reference to FIG. 3, the controller 160 (not shown, see FIG. 2) may deliver high frequency signals between a first pair of electrodes, e.g., active electrode 122a and reference electrode 122d, thereby creating a circuit path that includes the blood external to the first lead between the electrodes 122a, 122d. The other electrodes may then be activated in pairs, e.g., electrodes 122b, 122c, to detect the resistance of the fluid based upon the signals being delivered by the first pair of electrodes 122a, 122d. As the blood volume within the right ventricle 14 rises and falls, the electrical resistance varies, e.g., increasing as the fluid volume reduces, and decreasing as the fluid volume increases. The resistance detected by the pairs of electrodes 122 may be summed and recorded as a surrogate for the fluid volume within the right ventricle 14 at any point in time and used to approximate the fluid volume as a function of time.

Alternatively, the controller 160 may be used to deliver high frequency carrier signals to the pair of electrodes 122a, 122d. The carrier signals may be modulated as a result of the flow of blood into and out of the right ventricle 14. The signals may be demodulated by the controller 160, converted into digital signals, and processed to obtain impedance or resistance values. For example, the controller 160 may divide the resistance values into the product of blood resistivity and the square of the distance between the electrodes 122a, 122d, thereby providing a measure of the blood volume within the right ventricle 14. Additional information on methods for measuring impedance may be found in U.S. Pat. Nos. 4,674,518 and 5,417,717, the entire disclosures of which are expressly incorporated by reference herein.

The controller 160 may store the fluid volume data along with pressure data from the pressure sensor 120, e.g., as a function of time to determine the pressure-volume relationship for the right ventricle 14. For example, the controller 160 may generate one or more PV loops based upon the cardiac cycle of the heart based on the volume of the first chamber as a function of time and the measured pressure. The PV loops may allow the controller 160 to automatically ascertain certain information and modify pacing or other therapy to the heart 10 accordingly. For example, the controller 160 may determine when the right ventricle 14 is optimally filled with blood based upon the PV loops, and deliver electrical signals to the first pacing electrode 124 to cause contraction of the right ventricle 14 when the right ventricle 14 is optimally filled with blood.

Returning to FIG. 2, an exemplary method for implanting the system 100 will now be described. Although the delivery and/or implantation of the various components are described as being performed in an exemplary order, it will be appreciated that the components and steps may be performed in a different order than that described.

Initially, one or more leads may be delivered into the heart 10 of a patient. For example, the first lead 110 may be introduced into the patient's body, e.g., from a percutaneous puncture in a peripheral vessel, such as a subclavian vein, femoral vein, and the like (not shown), and advanced through the patient's vasculature into the heart 10, e.g., via the superior or inferior vena cava into the right atrium 12. Optionally, the first lead 110 may be delivered over a guidewire or other rail (not shown) and/or through a guide catheter (also not shown) that have been previously placed within the right atrium 12 and/or right ventricle 14 of the heart 10.

Once the distal end 114 of the first lead 110 is disposed within the right atrium 12, the distal end 114 may be directed through the tricuspid valve into the right ventricle 14, as shown in FIG. 14. The first pacing electrode 124 may be secured within the right ventricle 14, e.g., to the myocardium adjacent the right AV bundle 26 (see FIG. 1). As shown in FIG. 2, with the first pacing electrode 124 secured, the pressure sensor 120 and the resistance measuring electrodes 122 are also disposed within the right ventricle 14, e.g., when the tricuspid valve is closed. Also as shown in FIG. 2, it may be desirable to locate the pressure sensor 120 on the distal end 114 along the mid-portion of the resistance measuring electrodes 122, e.g., to ensure adequate exposure of the pressure sensor 120 to fluid pressure within the right ventricle 14. Alternatively, if one or more of the resistance measuring electrodes 122 are disposed within the right atrium 12 when the distal end 114 is fully advanced into the right ventricle 14, these electrodes 122 may be deactivated or ignored during use. These electrodes may be ignored automatically based upon analysis by the controller 160 or based upon instructions sent to the controller 160 by a clinician, e.g., after observing or monitoring delivery of the first lead 110.

Similarly, the second lead 130 may be introduced into the patient's vasculature and advanced into the right atrium 12. The distal end 134 of the second lead 130 may then be directed into the coronary sinus 13 and advanced through the venous system of the heart 10, e.g., until the second pacing electrode 144 is disposed adjacent the left ventricle 18. For example, the distal end 134 of the second lead 130 may be directed into the lateral coronary vein 19 (see FIG. 1), which may be disposed adjacent the left ventricle 18. The second pacing electrode 144 may be secured relative to the myocardium adjacent the left ventricle 18. For example, the second pacing electrode 144 may be screwed into tissue adjacent the lateral coronary vein 19, may be wedged into the lateral coronary vein 19, or may otherwise be secured, as described elsewhere herein.

Alternatively, the second lead 130 may be delivered directly into the left ventricle 18 (not shown). For example, the second lead 130 may be introduced from an entry site, through the patient's vasculature, and into the right atrium 12. After entering the right atrium 12, the second lead 130 may be directed through an atrial septostomy, which has been previously created using known procedures, into the left atrium 16, and then the distal end 134 may be advanced through the mitral valve into the left ventricle 18. In this alternative, the second pacing electrode 144 may be secured relative to the myocardium, e.g., by screwing the second pacing electrode 144 into the myocardium adjacent the left ventricle 18.

Similarly, the third lead 150 may be introduced into the patient's vasculature and advanced into the right atrium 12. The third pacing electrode 156 may then be secured to the wall of the right atrium 12, e.g., to provide a return path for electricity delivered by the first and second pacing electrodes 124, 144 through the walls of the heart 10.

The leads 110, 130, 150 may then be coupled to the controller 160. For example, as described elsewhere herein, the proximal ends 112, 132, 152 of the leads 110, 130, 150 may include connectors (not shown) that may be connected to mating connectors on the controller 160. If the controller 160 is to be implanted within the patient's body, e.g., subcutaneously, the controller 160 may be implanted, and the proximal ends 112, 132, 152 routed using conventional methods. Alternatively, if the controller 160 is located externally to the patient's body, the proximal ends 112, 132, 152 may be routed out of the patient's body to the controller 160, also using conventional methods.

Generally, the controller 160 may thereafter receiving pressure data from the pressure sensor 120 and resistance data from the plurality of electrodes 122, e.g., to determine a pressure-volume relationship for the right ventricle 14, as described elsewhere herein. The controller 160 may monitor the data and/or determine the pressure-volume relationship substantially continuously or periodically, as desired. In addition, the controller 160 may deliver electrical energy to one or more of the pacing electrodes 124, 144, 156, e.g., based at least in part upon the determined pressure-volume relationship for the right ventricle 14 to deliver electrical therapy to the heart 10. For example, the controller 160 may utilize an algorithm to assess the PV loop and adjust timing of the pacing pulses to the electrodes 124, 144, 156 according to the PV loop. For example, the controller 160 may analyze the PV loop to determine an appropriate sequence and/or interval between delivering pacing pulses to the first and second pacing electrodes 124, 144.

As an example, it may be desirable to have the right ventricle 14 contract as soon as the right ventricle 14 is substantially filled, and not before. The resistance measured in the right ventricle 14, acting as a surrogate for volume, may indicate when the desired ventricular volume has been achieved. The controller 160 may detect this event, and activate the pulse generator 166 to deliver pacing energy to the first pacing electrode 124, thereby causing the right ventricle 14 to contract.

Optionally, if the controller 160 includes a transceiver 174, the therapy may be adjusted by a clinician independent of existing algorithm(s) used by the controller 160. For example, data related to the pressure, fluid volume, and/or pressure-volume relationship may be transmitted via the transceiver 174 to an external device. A clinician may then analyze the data, and determine a new therapy plan for the patient, and direct the external device to provide appropriate instructions to the controller 160 via the transceiver 174. Thus, the existing algorithms may be replaced with new algorithms based upon the PV loop data obtained by the controller 160. For example, an external controller or programming device may be used to modify or replace the algorithms utilized by the controller 160. In an alternative embodiment, the controller 160 may be used simply to transmit pressure and resistance data, or pressure and fluid volume data via the transceiver 174, whereupon the pacing electrodes 122, pulse generator 166, and possibly other components of the system 100 may be eliminated.

Optionally, the controller 160 may allow one or more components to be disabled, e.g., by a clinician via an external controller. For example, if pacing of only the right ventricle 14 has been found to be effective, the controller 160 may discontinue delivery of pacing to the left ventricle 18, i.e., by shutting off the second pacing electrode 144. Similarly, pacing of the right ventricle 14 may be discontinued while pacing the left ventricle 18 continues.

Turning to FIG. 5, another embodiment of a system 100′ is shown that generally includes leads 110, 130,′ 150, and a controller 160.′ The first lead 110 may be similar to the embodiment shown in FIG. 2 and described elsewhere herein. The first lead 110 may also be delivered similar to the first lead shown in FIG. 2, e.g., placed via venipuncture, through the right atrium 12, and into the right ventricle 14. Similarly, the third lead 150 may be delivered and secured within the right atrium 12.

Unlike the previous embodiments, the second lead 130′ may include a pressure sensor 140′ and a second set of electrodes, e.g., a plurality of resistance measuring electrodes 142′ on the distal end 134,′ as well as a second pacing electrode 144.′ The second lead 130′ may be introduced from an entry site, through the patient's vasculature, and into the right atrium 12. After entering the right atrium 12, the second lead 130′ may be directed through an atrial septostomy, which has been previously created using known procedures, into the left atrium 16, and then the distal end 134′ may be advanced through the mitral valve into the left ventricle 18.

In this embodiment, the second pacing electrode 144′ may be secured relative to the myocardium, e.g., by screwing the second pacing electrode 144′ into the myocardium adjacent the left ventricle 18. Once the distal end 134′ is positioned within the left ventricle 18, the pressure sensor 140′ and the resistance measuring electrodes 142′ are disposed within the left ventricle 18, as shown in FIG. 5. Alternatively, if some of the resistance measuring electrodes 142′ are not located within the left ventricle 18, these electrodes may be deactivated or ignored, similar to the previous embodiments.

The three leads 110, 130,′ 150 may then be coupled to a controller 160′ similar to the previous embodiments. Generally, the controller 160′ may be constructed and operate similar to the embodiment shown in FIG. 4. However, unlike the previous embodiments, the controller 160′ may receive pressure data and resistance data from both ventricles 14, 18. Furthermore, the controller 160′ may determine PV loops for both ventricles 14, 18, which may be used to modify delivery of electrical energy to the pacing electrodes 124, 144,′ 156. In addition, if the controller 160′ includes a transceiver, data may be transmitted to a remote location and/or instructions may be received from an external controller, e.g., to modify therapy to both ventricles 14, 18 based upon the PV loops.

It will be appreciated that, in this embodiment, different frequencies may be used for the active and reference electrodes of the resistance measuring electrodes in each of the ventricles 14, 18 in order to avoid interference. For example, the controller 160′ may deliver signals to the active and reference electrodes of the first and second sets of resistance measuring electrodes 122, 142′ at different frequencies. In an exemplary embodiment, a frequency of about 1.3 kiloHertz (kHz) may be used for the active and reference electrodes of the first set of resistance measuring electrodes 122 on the first lead 110 and a frequency of about 1.6 kiloHertz (kHz) may be used for active and reference electrodes of the second set of electrodes 142′ on the second lead 130.′ The controller 160′ may include band pass filters for isolating the resistance signals obtained from the pairs of resistance measuring electrodes in each of the ventricles. Without the filters, signals within the right ventricle 14 may leak into the left ventricle 18 (and vice versa), which may prevent accurate determination of the resistance signals.

Alternatively, a single frequency generator within the controller 160′ may be used instead of multiple frequencies. In this alternative, the controller 160′ may alternate back and forth between the first and second sets of resistance measuring electrodes 122, 142.′ Thus, only one set of electrodes may be activated at a time, thereby preventing signals from one ventricle leaking into the other. In an exemplary embodiment, the controller 160′ may switch between the first and second sets about every twenty milliseconds (20 ms), and interpolate the resistance data obtained to approximate the fluid volume within each of the ventricles as a function of time.

Turning to FIG. 6, an exemplary idealized PV loop, ABCD, is shown for a single cycle of a left ventricle of a heart, and an exemplary actual PV loop, A′B′C′D,′ for a diseased heart. Generally, the cycle of the left ventricle includes four basic phases. The right ventricle behaves generally in a similar manner. At point A of the idealized PV loop, the mitral valve may open, and between A-B, the left ventricle may begin to fill (diastole). At point B, the left ventricle begins to contract isovolumetrically between B-C, i.e., with the aortic valve (and other valves) closed. At point C, once the aortic diastolic pressure is exceeded, the aortic valve opens, and the blood is ejected from the left ventricle between C-D (systole). Finally, at point D, the aortic valve closes, and the left ventricle relaxes isovolumetrically between D-A, whereupon the process repeats itself, generating another PV loop.

One particularly useful characteristic of the PV loop is “end-systolic elastance,” which is the end-systolic pressure volume relationship (“ESPVR”) identified by line E in FIG. 6. The slope of this line may communicate information to a clinician regarding the overall performance of the heart. In addition, the area of the PV loop represents the stroke work, which is the work of the heart during each heart beat. Stroke volume is equal to the end-diastolic volume minus the end-systolic volume, which is the amount of blood ejected from the left ventricle out of the heart with each heart beat. Heart fraction is related to the stroke volume except that it is recited as a percentage, i.e., the ratio of the stroke volume to the total volume. For example, if the left ventricle ejects at least about fifty five percent (55%) of the total volume of blood within the left ventricle per heart beat, the heart fraction may indicate good heart function. One or more of these characteristics of the heart may be determined by the controller 160′ for one or both ventricles of the heart, e.g., in real time.

By generating PV loops, the controller 160′ and system 100′ may effectively determine these phases of the heart's cycle in real time, and/or deliver pacing energy to modify the cycle of the heart and/or otherwise operate the heart more efficiently. The PV loops may also allow the slopes of the phases and/or other useful points to be determined, such as peak systolic pressure (the highest point between C-D), end-systolic elastance, and/or ejection fraction. The controller 160′ may be programmed with one or more algorithms to modify pacing therapy based upon the data obtained and/or to transmit the data to a clinician who may then reprogram or modify the controller 160′ based upon analysis of the data.

Over time, the PV loops of the heart may be modified in a desired manner. For example, various conditions may cause the PV loops to deviate from normal, healthy shapes into other less efficient shapes. For example, PV loop A′B′C′D′ shown in FIG. 6 may indicate dilated cardiomyopathy. This condition is characterized by dilatation and impaired contractility of the left ventricle, and may cause the PV loop for the left ventricle to shift right and down (relative to the idealized PV loop ABCD shown in FIG. 6). Thus, pacing therapy to such a dilated heart may be modified to adjust the shape of this PV loop.

Other conditions that may be identified, monitored, and/or considered when modifying pacing therapy include hypertrophic cardiomyopathy, characterized by left ventricular hypertrophy, which may cause increased left ventricular wall thickness, and restrictive cardiomyopathy, which is characterized by increased diastolic stiffness of the left ventricle. With the first condition, the PV loop may shift left, and the ESPVR may shift left and upward. The results of these conditions may be a lower total area as the PV loop is compressed, reducing stroke work, stroke volume, and other aspects of heart function. Thus, analysis of the PV loops of the heart over time may facilitate analysis, identification, and determining proper course of pacing or other treatment.

In addition, the PV loop may provide other insight into the condition of the heart. For example, as shown in FIG. 6, point B′ includes a slight overshoot in volume before isovolumetric contraction, which may indicate valvular disease. Thus, the transitions between the phases may indicate prolapse, regurgitation, and the like. Monitoring PC loops of a patient's heart during various activities may provide insight into the ability of the heart to operate during various levels of activity, while being treated with various pharmaceuticals, or other pathological analysis.

In other embodiments, one or more of the features described herein may be coupled with cardioversion and defibrillation capability, including the ability to sense ventricular tachycardia or fibrillation and delivery either pacing or defibrillation energy as indicated. In addition, the systems and methods described herein may be used to analyze heart function for diagnostic purposes either alone or in conjunction with other analytical tools. In addition, data from the PV loops may also be used to monitor effects of other interventions, such as pharmacologic interventions.

In another embodiment, one or more leads or catheters and a controller may be used simply as a recorder and/or communicator, e.g., for storing data related to the PV loops of one or both ventricles. The data may be transmitted to a remote location for diagnostic analysis and/or treatment of the patient. Thus, the pacing electrodes may be eliminated and the controller components related to pacing may also be omitted.

It will be appreciated that elements or components shown with any embodiment herein are exemplary for the specific embodiment and may be used on or in combination with other embodiments disclosed herein. In addition, it will be appreciated that the methods described herein may be applicable to other devices in addition to implantable leads. For example, catheters or other devices may include light sensitive material that may be activated to modify the stiffness in a desired manner.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A pacemaker system for pacing a heart of a patient, comprising:
a first lead comprising a first proximal end, a first distal end sized for introduction into a body lumen, a pressure sensor on the first distal end for measuring pressure within a first chamber of a heart within which the first distal end is delivered, a first set of electrodes on the first distal end for measuring electrical resistance of fluid within the first chamber, and a first pacing electrode for delivering electrical energy to tissue adjacent the first chamber;
a second lead comprising a second proximal end, a second distal end sized for introduction into a body lumen, and a second pacing electrode on the second distal end for delivering electrical energy to tissue adjacent a second chamber of a heart; and
a controller coupled to the first and second proximal ends, the controller receiving pressure data from the pressure sensor and resistance data from the first set of electrodes for determining a pressure-volume relationship for the first chamber, the controller comprising a pulse generator for delivering electrical energy to at least one of the first and second pacing electrodes based at least in part upon the pressure-volume relationship for the first chamber to deliver electrical therapy to the heart.

2. The system of claim 1, wherein the controller is configured for modifying at least one of the following based at least in part upon the pressure-volume relationship for the first chamber: a sequence of delivering electrical energy to the first and second pacing electrodes, a delay between delivering electrical energy to the first and second pacing electrodes, and a duration of delivering electrical energy to the first and second pacing electrodes.

3. The system of claim 1, further comprising a third lead comprising a third proximal end coupled to the controller, a third distal end sized for introduction into a body lumen, and a third pacing electrode on the third distal end for delivering electrical energy to tissue adjacent a third chamber of a heart, the pulse generator configured for delivering electrical energy to the third pacing electrode based at least in part upon the pressure-volume relationship for the first chamber to deliver electrical therapy to the heart.

4. The system of claim 1, wherein the second lead further comprises a second set of electrodes on the second distal end for measuring resistance of fluid within the second chamber.

5. The system of claim 4, wherein the second lead further comprises a pressure sensor on the second distal end for measuring pressure within the second chamber, the controller configured for receiving pressure data from the pressure sensor and resistance data from the plurality of electrodes for determining a pressure-volume relationship for the second chamber, the controller further configured for delivering electrical energy to the first and second pacing electrodes based at least in part upon the pressure-volume relationship for the second chamber to deliver electrical therapy to the heart.

6-9. (canceled)

10. The system of claim 1, the controller comprising a processor for generating a pressure-volume loop based upon the cardiac cycle of the heart within which the first and second leads are delivered, the controller controlling the pulse generator for adjusting electrical therapy based upon the pressure-volume loop.

11. The system of claim 10, wherein the processor is configured for relating the measured resistance to volume of the first chamber as a function of time and generating the pressure-volume loop based at least in part upon the volume of the first chamber as a function of time.

12. The system of claim 10, wherein the processor is configured for determining when the first chamber is optimally filled with blood, the controller controlling the pulse generator to activate the first pacing electrode to cause contraction of the first chamber when the processor determines the first chamber is optimally filled with blood.

13. The system of claim 1, further comprising a transmitter coupled to the controller for transmitting data including at least one of the measured pressure, the measured resistance, and the pressure-volume relationship for the first chamber to a location external to the patient.

14. The system of claim 13, further comprising a receiver coupled to the controller for receiving instructions from a location external to the patient, the controller configured for controlling the pulse generator based at least in part upon the instructions to adjust the electrical therapy of the heart.

15-17. (canceled)

18. A method for biventricular pacing using first and second leads comprising one or more electrodes implanted within a heart for delivering electrical signals to tissue adjacent first and second chambers, respectively, of the heart, the method comprising:
measuring pressure within the first chamber using the first lead;
measuring electrical resistance of fluid within the first chamber using the first lead;
determining a pressure-volume relationship for the first chamber based upon the pressure and resistance measured within the first chamber; and
delivering electrical signals to one or more electrodes on the first and second leads based at least in part upon the pressure-volume relationship for the first chamber to provide electrical therapy to the heart.

19. The method of claim 18, wherein determining a pressure-volume relationship for the first chamber comprises:
relating the measured resistance to volume of the first chamber as a function of time; and
generating a pressure-volume loop based upon the cardiac cycle of the heart based at least in part on the volume of the first chamber as a function of time and the measured pressure.

20. The method of claim 19, wherein determining a pressure-volume relationship for the first chamber further comprises determining when the first chamber is optimally filled with blood based upon the pressure-volume loop, and wherein delivering electrical signals comprises activating one or more electrodes on the first lead to cause contraction of the first chamber when the processor determines the first chamber is optimally filled with blood.

21. The method of claim 18, further comprising:
measuring pressure within the second chamber using the second lead; and
measuring electrical resistance of fluid within the second chamber using the second lead; and
determining a pressure-volume relationship for the second chamber based upon the pressure and resistance measured within the second chamber.

22-24. (canceled)

25. The method of claim 18, wherein the first chamber comprises a right ventricle of the heart, and wherein a distal end of the first lead is delivered within the right ventricle to measure pressure and resistance of fluid within the right ventricle.

26. The method of claim 25, wherein the second chamber comprises a left ventricle of the heart.

27-28. (canceled)

29. A method for implanting a biventricular pacing system within a heart of a patient, comprising:
delivering a distal end of a first lead through the patient's vasculature into a first chamber of the heart such that a pressure sensor and a plurality of electrodes on the distal end are disposed within the first chamber;
securing a first pacing electrode on the distal end of the first lead to the myocardium adjacent the first chamber;
delivering a distal end of a second lead through the patient's vasculature into the heart;
securing a second pacing electrode on the distal end relative to the myocardium adjacent a second chamber of the heart; and
coupling the first and second leads to a controller, the controller configured for receiving pressure data from the pressure sensor and resistance data from the plurality of electrodes to determine a pressure-volume relationship for the first chamber, the controller comprising a pulse generator for delivering electrical signals to at least one of the first and second pacing electrodes based at least in part upon the determined pressure-volume relationship for the first chamber to deliver electrical therapy to the heart.

30-36. (canceled)

37. An implantable device for determining the pressure-volume relationship of a first chamber of a heart, comprising:
an elongate member comprising a proximal end, a distal end sized for introduction into a first chamber of a heart, a pressure sensor on the distal end for measuring pressure within the first chamber, and a resistance sensor for measuring fluid electrical resistance within the first chamber;
a processor coupled to the proximal end of the elongate member for obtaining pressure data from the pressure sensor and fluid resistance data from the resistance sensor, the processor configured for determining fluid volume data comprising the volume of fluid within the first chamber and for determining a pressure-volume relationship for the first chamber based upon the pressure data and the fluid volume data.

38. The device of claim 37, further comprising an alternating current source coupled to the processor for delivering electrical signals to the resistance sensor, and voltage measuring means for measuring the fluid resistance based at least in part on the electrical signals delivered to the resistance sensor.

39. The device of claim 37, wherein the elongate member comprises a catheter.

40. The device of claim 37, wherein the elongate member comprises a lead, the lead further comprising one or more pacing electrodes on the distal end.

41. The device of claim 37, wherein the resistance sensor comprises a plurality of electrodes spaced apart from one another along the distal end such that the plurality of electrodes are disposed within the first chamber when the distal end is delivered into the first chamber.

42. The device of claim 37, wherein the processor is disposed within a controller, the controller sized for implantation within a patient's body.
